(19) United States
(12) Patent Application Publication
(10) Pub. No.: US 2026/0027065 A1
(43) Pub. Date: Jan. 29, 2026

(54) LIVER/ADIPOSE TISSUE DUAL-TARGETING COMPOSITE NANO-DRUG CARRIER

(71) Applicant: IUCF-HYU (INDUSTRY-UNIVERSITY COOPERATION FOUNDATION HANYANG UNIVERSITY), Seoul (KR)

(72) Inventors: Yong-Hee KIM, Seoul (KR); Ju Hyeong HONG, Seoul (KR)

(73) Assignee: IUCF-HYU(INDUSTRY-UNIVERSITY COOPERATION FOUNDATION HANYANG UNIVERSITY), Seoul (KR)

(21) Appl. No.: 18/997,945

(22) PCT Filed: Jul. 26, 2023
(86) PCT No.: PCT/KR2023/010782

§ 371 Date: Jan. 23, 2025

(30) Foreign Application Priority Data
Jul. 26, 2022 (KR) 10-2022-0092319

(51) Int. Cl. A61K 9/51; A61K 31/555; A61P 3/04

(52) U.S. Cl. CPC A61K 9/5153; A61K 9/5169; A61K 9/5192; A61K 31/555; A61P 3/04

(57) ABSTRACT

The present invention relates to: a liver/adipose tissue dual-targeting composite nano-drug carrier comprising drug-containing poly(L-lactide-co-glycolide) (PLGA) nanoparticles and an adipocyte targeting sequence (ATS) peptide that can target prohibitin; a method for preparing same; and uses thereof for medicines and health foods for preventing or treating obesity or obesity-induced metabolic diseases.

### TECHNICAL FIELD

The present invention relates to a liver/adipose tissue dual-targeting composite nano-drug carrier, a method of preparing the same, and a use thereof.

### BACKGROUND ART

Many drugs currently used as pharmaceuticals are insoluble and exhibit low bioavailability when administered to the body due to low solubility, and a considerable amount of drug candidates that are under development are also difficult to formulate due to poor solubility. Therefore, many different formulation methods have been studied to solubilize poorly soluble drugs, but their effects have been minimal or their application has been limited to date. Polymer nanoparticles are one of the important fields in drug delivery systems, and recently, a variety of research has been conducted on the production of nanoparticles using amphiphilic polymers.

Polymer nanoparticles are one of the important fields in drug delivery systems. For synthetic polymers, poly(lactide-co-glycolide) (PLGA), which is a copolymer, has been used as a sustained-release drug formulation for decades, and in addition to high biocompatibility and a controllable biodegradation ability, it has the advantages of having different biodegradation mechanisms depending on the composition of constituent monomers, such as lactide and glycolide, and the environment, and ultimately being released from the body through metabolism after being degraded and converted into non-toxic small molecules. In addition, PLGA has been widely used as a preparation for tissue engineering or intraperitoneal administration, mainly in a form with a size distribution of microns or larger.

Heme oxygenase-1 (HO-1) is a heme breakdown protein that induces expression from oxidative stress in vivo, and has been reported to have major functions such as antioxidation, antiinflammation, inhibition of immune responses, cell survival, and angiogenesis.

Particularly, a HO-1 inducer induces energy production by decomposing fatty acids in adipocytes, and inhibits an inflammatory response in adipose tissue through differentiation of adipose tissue-derived macrophages to anti-inflammatory macrophages. In addition, the HO-1 inducer inhibits the destruction of liver cells by fatty acids and inhibits the inflammatory response in liver tissue by differentiating liver tissue-derived macrophages into anti-inflammatory macrophages. This can promote the treatment of obesity, obesity-induced type 2 diabetes, and non-alcoholic steatohepatitis by simultaneously inducing the inhibition of fatty acid accumulation and anti-inflammatory response.

Current therapeutic agents for obesity and diabetes have serious side effects caused by hormone-based treatments, such as appetite suppression mechanisms, and are insufficient to directly treat diseased tissue. Therapeutic agents for fatty liver disease are only partial treatments for liver tissue and have little long-term effect. In addition, in the case of existing carriers, the problem of target transmission was raised.

Accordingly, drug delivery systems are needed to maximize treatment so that the solubilized and released poorly soluble drugs which are effective against obesity or obesity-induced metabolic diseases, such as HO-1 inducers, can be delivered to a desired site.

### RELATED ART DOCUMENT

(Non-patent document 1) Tu, T. H., Joe, Y., Choi, H. S., Chung, H. T., & Yu, R. (2014). Induction of Heme Oxygenase-1 with Hemin Reduces Obesity-Induced Adipose Tissue Inflammation via Adipose Macrophage Phenotype Switching. Mediators of Inflammation, 2014, 1-10.

### DISCLOSURE

### Technical Problem

To solve the problems of existing drug carriers and existing therapeutic agents for obesity or an obesity-induced metabolic disease, much effort went into research on manufacturing dual-targeting drug carriers which can target both adipose tissue and liver tissue so that prohibitin receptors are overexpressed on the cell membranes of adipocytes in adipose tissue and adipose tissue-derived macrophages, liver cells, and liver tissue-derived macrophages when fatty acids are excessively accumulated in the liver and adipose tissue and drugs can be specifically delivered using these prohibitin receptors, leading to development of composite drug carriers in which a poorly soluble drug effective for obesity or obesity-induced metabolic diseases, such as an HO-1 inducer, and a dual-targeting material capable of targeting both adipose tissue and liver tissue is combined with a drug carrier. Thus, the present invention was completed.

### Technical Solution

Therefore, the present invention is directed to providing a liver/adipose tissue dual-targeting composite nano-drug carrier, which includes an adipocyte targeting sequence (ATS) peptide capable of targeting drug-containing poly(L-lactide-co-glycolide) (PLGA) nanoparticles and prohibitin.

The present invention is also directed to providing a method of preparing a liver/adipose tissue dual-targeting nano-drug carrier, which includes combining an ATS peptide with drug-containing PLGA composite nanoparticles using linkers.

The present invention is also directed to providing a composition for preventing or treating obesity or an obesity-induced metabolic disease, which includes the composite nano-drug carrier as an active ingredient.

The present invention is also directed to providing a food composition for preventing or improving obesity or an obesity-induced metabolic disease, which includes the composite nano-drug carrier or a nano-drug carrier prepared by the above-described method as an active ingredient.

### Advantageous Effects

Currently targeted treatments for obesity, diabetes, or fatty acid diseases include only appetite suppressants that act on the central nervous system or drugs that reduce fat accumulation or suppress inflammatory responses in liver tissue, and no therapeutic agents have been reported to treat metabolic diseases, including obesity, by simultaneous effects on adipocytes, immune cells, and liver cells, such as the drug carrier of the present invention.

Accordingly, the composite nano-drug carrier according to the present invention is expected to be very useful in the treatment of metabolic diseases including obesity by simultaneous effects on adipocytes, immune cells, and liver cells, since it enables direct treatment of diseased tissue and dual targeting of liver tissue and adipose tissue.

### Description of Drawings

FIG. 1 is a schematic diagram of ATS/PLGA NPs manufactured according to one embodiment of the present invention.

FIG. 2 shows the H-NMR result for the ATS/PLGA NPs manufactured according to one embodiment of the present invention [covalent bonding at each part of FIG. 1 can be confirmed].

FIG. 3 shows the result that confirms drug-specific delivery ability according to a linker molecular weight and the mixed ratio of a linker/PLGA NPs.

FIG. 4 shows the result that shows the zeta potential and nanoparticle size of the ATS/PLGA NPs manufactured according to one embodiment of the present invention.

FIG. 5 shows the result that shows the drug release amount of the ATS/PLGA NPs manufactured according to one embodiment of the present invention.

FIG. 6 shows the result of the time-dependent long-term distribution of ATS/PLGA NPs in animal models of obesity-induced type 2 diabetes.

FIG. 7 shows the result showing that prohibitin is overexpressed in the fatty liver of animal models of obesity-induced non-alcoholic steatohepatitis.

FIG. 8 shows the results of the time-dependent long-term distribution of ATS/PLGA NPs in animal models of obesity-induced non-alcoholic steatohepatitis.

FIG. 9 shows the results of evaluating the change in body weight by week after 4-week administration of ATS/PLGA NPs and insulin resistance 4 weeks after the end of administration in animal models of obesity-induced type 2 diabetes.

FIG. 10 shows the result of analyzing biomarkers in adipose tissue after 4-week administration of ATS/PLGA NPs in animal models of obesity-induced type 2 diabetes.

FIG. 11 shows the result of measuring the change in body weight by week after 4 weeks of administration of ATS/PLGA NPs in animal models of obesity-induced non-alcoholic steatohepatitis.

FIG. 12 shows the result of analyzing biomarkers in adipose tissue after 4-week administration of ATS/PLGA NPs in animal models of obesity-induced non-alcoholic steatohepatitis.

FIG. 13 shows the result of analyzing biomarkers in liver tissue after 4-week administration of ATS/PLGA NPs in animal models of obesity-induced non-alcoholic steatohepatitis.

FIG. 14 shows the results of analyzing M2 phenotype differentiation of adipose tissue macrophages after 4-week administration of ATS/PLGA NPs in animal models of obesity-induced type 2 diabetes.

FIG. 15 shows the results of analyzing M2 phenotype differentiation of adipose tissue macrophages after 4-week administration of ATS/PLGA NPs in animal models of obesity-induced non-alcoholic steatohepatitis.

FIG. 16 shows the results of analyzing blood lipid composition after 4-week administration of ATS/PLGA NPs in animal models of obesity-induced type 2 diabetes.

FIG. 17 shows the results of analyzing lipid composition in liver tissue after 4-week administration of ATS/PLGA NPs in animal models of obesity-induced non-alcoholic steatohepatitis.

FIG. 18 shows the results of analyzing the amount of inflammatory cytokine proteins in adipose tissue (top) and blood (bottom) after 4-week administration of ATS/PLGA NPs in animal models of obesity-induced type 2 diabetes.

FIG. 19 shows the results of analyzing the expression levels of inflammatory cytokines in liver tissue after 4-week administration of ATS/PLGA NPs in animal models of obesity-induced non-alcoholic steatohepatitis.

FIG. 20 shows the results of analyzing the expression levels of biomarkers (TGF-beta, IFNr, aSMA, and hydroxyproline) associated with the development of cirrhosis in liver tissue after 4-week administration of ATS/PLGA NPs in animal models of obesity-induced non-alcoholic steatohepatitis.

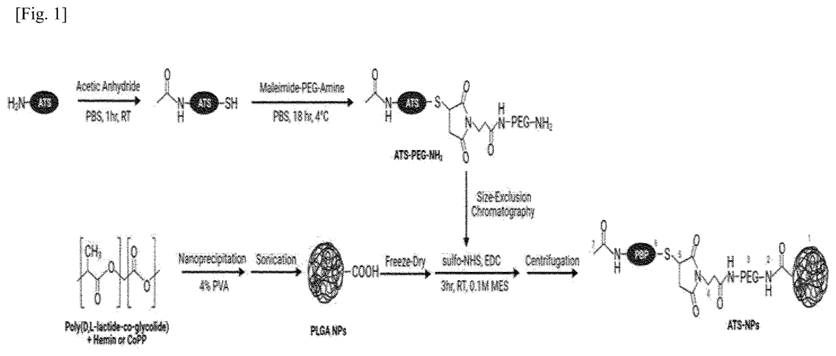

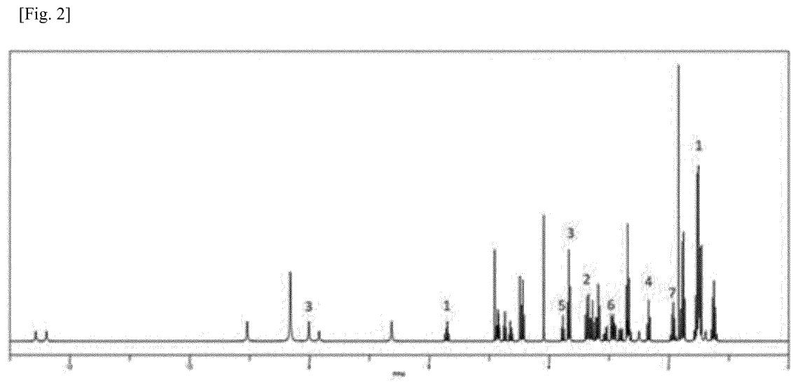

### Modes of the Invention

The present invention relates to a liver/adipose tissue dual-targeting composite nano-drug carrier, which includes an adipocyte targeting sequence (ATS) peptide capable of targeting drug-containing poly(L-lactide-co-glycolide) (PLGA) nanoparticles and prohibitin.

Detailed description of the present invention will now follow.

“Drug” used herein refers to a poorly soluble drug, and a heme oxygenase-1 (HO-1) inducer that can treat obesity or an obesity-induced metabolic disease.

The HO-1 inducer induces energy production by decomposing fatty acids in adipocytes and suppresses inflammatory responses in adipose tissue by differentiating adipose tissue-derived macrophages into anti-inflammatory macrophages. In addition, it suppresses the destruction of liver cells by fatty acids and suppresses inflammatory responses in liver tissue by differentiating liver tissue-derived macrophages into anti-inflammatory macrophages. This can promote the treatment of obesity, obesity-induced type 2 diabetes, and obesity-induced non-alcoholic steatohepatitis by simultaneously inducing the inhibition of fatty acid accumulation and anti-inflammatory responses.

The HO-1 inducer may specifically be cobaltic protoporphyrin IX chloride (CoPP) or hemin, but the present invention is not limited thereto.

“Adipocyte targeting sequence (ATS) peptide” used herein refers to Cys Lys Gly Gly Arg Ala Lys Asp Cys (CKGGRAKDC; SEQ ID NO: 1) that can target prohibitin.

It is known that prohibitin is highly expressed in adipose tissue, and in a study that preceded the present invention, it was revealed that the ATS peptide sequence can bind to prohibitin of white adipose cells and target adipose cells, particularly macrophages in visceral adipose cells. Macrophages in visceral fat play a key role in the inflammatory response of obesity-induced metabolic syndrome. In addition, the present invention is significant in that it reveals that the same sequence can also target fatty liver, that is, liver tissue.

In one embodiment of the present invention, a composite nano-drug carrier in which an ATS peptide binds to the surface of drug-containing poly(L-lactide-co-glycolide) (PLGA) nanoparticles using linkers is provided.

The ATS peptide is in a form that contains Cys at both ends, exposing a thiol group. A linker is needed to bind it to the PLGA core later.

In the present invention, as a polymer core nano-drug carrier that encapsulates a poorly soluble drug, poly(L-lactide-co-glycolide) (PLGA) is preferably used, and to obtain the maximum encapsulation efficiency, PLGA more preferably has an average molecular weight of 5,000 to 18,000.

Maleimide-PEG-NH₂-modified ATS peptide binds to drug-encapsulated polymer PLGA nanoparticles in a molar ratio of 1:0.5 to 2 or 1:0.5 to 1.5, and when the ratio exceeds the above range, the standard deviation of the average particle size of the composite nano-drug carrier increases, making it difficult to secure uniformity in preparation.

The present invention also provides a method of preparing a liver/adipose tissue dual-targeting composite nano-drug carrier, which includes combining an ATS peptide with drug-containing PLGA nanoparticles using linkers.

In one embodiment of the present invention, the present invention may provide a method of preparing a liver/adipose tissue dual-targeting nano-drug carrier, which includes:

preparing drug-containing PLGA nanoparticles by dissolving the drug and PLGA, stirring the resulting mixture, and adding a PVA aqueous solution thereto;

preparing the ATS peptide modified with maleimide-PEG-amine by acetylating the N-terminal of the ATS peptide and reacting it with the maleimide-PEG-amine; and

reacting the drug-containing PLGA nanoparticles through an NHS/EDC substitution reaction, and reacting the resulting substitution with the maleimide-PEG-amine-modified ATS peptide at room temperature for 2 to 4 hours to combine the ATS peptide with the drug-containing PLGA nanoparticles using linkers.

The concentration of the PVA aqueous solution may be 3 to 5% (w/v) to obtain the maximum encapsulation efficiency.

The ATS peptide is protected by acetylating the N-terminus of the peptide by reaction with acetic anhydride at room temperature. That is, an amine group is blocked, and without this process, the peptide is directly bound to the PLGA nanoparticles (PLGA NPs). In this case, PEG becomes the outermost layer of the nanoparticle, making cell targeting impossible. Afterwards, it is reacted with maleimide-PEG-NH₂ to form a thioester bond between maleimide and a thiol group, thereby preparing the maleimide-PEG-amine-modified ATS peptide.

The drug-containing PLGA nanoparticles are subjected to NHS/EDC substitution, and reacted with the maleimide-PEG-amine-modified ATS peptide at room temperature for 2 to 4 hours.

The drug-encapsulated polymer PLGA nanoparticles are reacted with the maleimide-PEG-amine-modified ATS peptide in a molar ratio of 1:0.5 to 2 to bind the PLGA nanoparticle-ATS peptide.

All of the above-described matters relating to the composite nano-drug carrier may be applied as is or applied mutatis mutandis to the method of preparing a composite nano-drug carrier.

The composite nano-drug carrier according to the present invention may specifically deliver a heme oxygenase-1 inducer enabling a potent anti-inflammatory action to adipose tissue and liver tissue, thereby inducing an anti-inflammatory effect in each tissue, and an effect of improving lipid metabolism and inhibiting lipid accumulation, and thus prevent or treat obesity or an obesity-induced metabolic disease.

Accordingly, the present invention provides a composition for preventing or treating obesity or an obesity-induced metabolic disease, which includes the composite nano-drug carrier as an active ingredient.

Examples of the obesity-induced metabolic disease include diseases that mainly occur when there is an abnormality in the hormones, liver, kidneys, etc., involved in regulating metabolism, and the types of the metabolic diseases may include, but are not limited to, obesity, diabetes, fatty liver disease, vascular disease, impaired glucose tolerance, hyperinsulinemia, and hyperglycemia. The vascular disease may be, but is not limited to, hypercholesterolemia, hyperlipidemia, hypertension, atherosclerosis, thrombosis, arteriosclerosis, hypertension, angina, myocardial infarction, ischemic heart disease, heart failure, vascular restenosis, cerebral infarction, cerebral hemorrhage, or stroke, and preferably, atherosclerosis. The fatty acid disease may be, but is not limited to, alcoholic fatty liver disease, non-alcoholic fatty liver disease, or non-alcoholic steatohepatitis, and preferably, non-alcoholic fatty liver disease.

The pharmaceutical composition of the present invention may be administered along with a pharmaceutically acceptable carrier, and for oral administration, the composition may further include a binder, a lubricant, a disintegrant, an excipient, a solubilizer, a dispersant, a stabilizer, a suspending agent, a colorant, and a flavoring, in addition to the active ingredient. For injections, the pharmaceutical composition of the present invention may be used by mixing a buffer, a preservative, an analgesic, a solubilizer, an isotonic agent, and a stabilizer. In addition, for local administration, the composition of the present invention may use a base, an excipient, a lubricant, and a preservative.

The pharmaceutical composition of the present invention may be prepared in various forms by being mixed with the above-described pharmaceutically acceptable carrier. For example, for oral administration, the pharmaceutical composition of the present invention may be prepared in various dosage forms such as a tablet, a troche, a capsule, an elixir, a suspension, a syrup and a wafer, and for injections, the pharmaceutical composition or vaccine composition of the present invention may be prepared in a unit dose ampoule or multiple dose forms. In addition, the pharmaceutical composition of the present invention may be formulated as a solution, a suspension, tablets, pills, capsules, or a sustained-release preparation.

Meanwhile, examples of carriers, excipients and diluents suitable for preparation may include lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate, and mineral oil. The examples of carriers, excipients and diluents may also include a filler, an anti-agglomerate, a glidant, a wetting agent, a flavoring, and a preservative.

The pharmaceutical composition of the present invention can be administered orally or parenterally. The composition according to the present invention may be administered, for example, orally, by an aerosol, buccally, transdermally, intradermally, by inhalation, intramuscularly, intranasally, intraocularly, intrapulmonarily, intravenously, intraperitoneally, intranasally, ocularly, intra-aurally, by injection or patches, subcutaneously, sublingually, topically, or percutaneously, but the present invention is not limited thereto.

For the above-mentioned clinical administration, the pharmaceutical composition of the present invention may be formulated in a suitable dosage form using a known technique. For example, for oral administration, it may be mixed with an inert diluent or edible carrier, sealed in hard or soft gelatin capsules, or pressed into tablets. For oral administration, the active ingredient may be mixed with excipients and used in the form of ingestible tablets, buccal tablets, troches, capsules, an elixir, a suspension, a syrup, or a wafer. In addition, various formulations for injection or parenteral administration may be prepared according to known or commonly used techniques in the related art.

An effective dose of the pharmaceutical composition of the present invention may vary according to a patient's weight, age and sex, a health condition, a diet, an administration time, an administration method, an excretion rate and the severity of a disease, and may be easily determined by a general expert in the related art.

The preferable dosage of the pharmaceutical composition of the present invention may be appropriately selected by those of ordinary skill in the art depending on a patient's condition and body weight, the severity of a disease, a drug type, an administration route and an administration duration. However, the pharmaceutical composition of the present invention is preferably administered daily at 0.001 to 100 mg/kg of body weight, and more preferably, 0.01 to 30 mg/kg of body weight. The administration may be performed once a day, or several times in divided portions. The composite nano-drug carrier of the present invention may be present at 0.0001 to 10 wt %, and preferably 0.001 to 1 wt % with respect to the total weight of the entire composition.

The pharmaceutical composition of the present invention may be administered to mammals such as mice, rats, livestock, and humans via various routes. There are no restrictions on the administration method, and for example, it can be administered orally, rectally, or by intravenous, intramuscular, subcutaneous, intrauterine epidural, or intracerebroventricular injection.

Accordingly, the present invention includes a method of preventing or treating obesity or an obesity-induced metabolic disease, which includes administering a therapeutically effective amount of the pharmaceutical composition including the composite to a subject.

The prevention or treatment method of the present invention includes administering a therapeutically effective amount of the composition of the present invention. The therapeutically effective amount is an amount that enhances the effect of suppressing obesity or an obesity-induced metabolic disease. It is self-evident to one of ordinary skill in the art that the appropriate total daily dose can be determined by a physician's reasonable medical judgement. It is preferable to apply the specific therapeutically effective amount for a specific patient differently depending on the type and degree of response to be achieved, a specific type of composition depending on the use of a different agent in some cases, the patient's age, weight, general health, sex, and diet, an administration time, an administration route, the secretion rate of the composition, the duration of treatment, and various factors, and similar factors well known in the medical field. Therefore, the effective amount of the pharmaceutical composition, which is suitable for the purpose of the present invention, is preferably determined by considering the above-mentioned matters. In addition, in some cases, the therapeutic effect of the related disease can be improved by co-administering a known therapeutic agent for the related disease together with the composition of the present invention.

The term “subject” of the present invention includes mammals such as horses, sheep, pigs, goats, camels, antelopes, or dogs, or humans, which have a related disease whose symptoms can be improved by administration of the pharmaceutical composition according to the present invention.

As another aspect of the present invention, the present invention provides a health functional food composition for preventing or improving obesity or an obesity-induced metabolic disease, which includes the composite nano-drug carrier as an active ingredient.

The term “improvement” used herein means any action that at least reduces the degree of parameters related to the condition being treated, such as the obesity index and pain related to a metabolic disease.

The term “health functional food” used herein is the same as a specific health food, and refers to a food with high medical and therapeutic effects that is processed to efficiently exhibit a bioregulatory function in addition to nutritional supply. In some cases, terms such as “functional food,” “health food,” and “health supplement” may be permitted, and the food may be manufactured in various forms such as tablets, capsules, powder, granules, a liquid, and pills to obtain a useful effect.

The health functional food of the present invention may be an additional component that is commonly used in a food composition to enhance its smell, taste, and appearance. For example, the health functional food of the present invention may include vitamins A, C, D, E, B1, B2, B6, and B12, niacin, biotin, folate, and pantothenic acid. In addition, the health functional food of the present invention may include minerals such as zinc (Zn), iron (Fe), calcium (Ca), chromium (Cr), magnesium (Mg), manganese (Mn), and copper (Cu). In addition, the health functional food of the present invention may include amino acids such as lysine, tryptophan, cysteine, and valine. The health functional food of the present invention may also include food additives, for example, a preservative (e.g., potassium sorbate, sodium benzoate, salicylic acid, or sodium dehydroacetate), a disinfectant (e.g., bleach powder, high-purity bleach powder, or sodium hypochlorite), an antioxidant (e.g., butylhydroxyanisole (BHA) or butylated hydroxy toluene (BHT)), a pigment (e.g., a tar pigment), a colorant (e.g., sodium nitrite), a bleaching agent (e.g., sodium sulfite), a flavor enhancer (e.g., monosodium glutamate (MSG)), a sweetener (e.g., dulcin, cyclamate, saccharin, or sodium), a flavoring (e.g., vanillin or lactone), a leavening agent (e.g., alum or D-potassium hydrogen tartrate), a strengthener, an emulsifier, a thickener (a gelling agent), a coating agent, a gum base, an antifoaming agent, a solvent, and a conditioner. These additives may be selected according to the type of food and used in appropriate amounts.

When the health functional food of the present invention is used as a food additive, it may be added alone or used in combination with another food or food component, and may be used appropriately by a conventional method.

In the health functional food of the present invention, the content of the composite nano-drug carrier is not particularly limited, and may vary depending on the condition of a subject receiving administration, the particular type of disease, and the degree of progression. If needed, the composite nano-drug carrier may also be included in the total content of the food.

Hereinafter, the present invention will be described in detail with reference to the following examples. The examples are merely provided to illustrate the present invention, but the scope of the present invention is not limited to the following examples. These examples are merely provided to complete the disclosure of the present invention and fully convey the scope of the present invention to those of ordinary skill in the art, and the present invention should be defined only by the accompanying claims.

### EXAMPLES

### Preparation Example 1: Preparation of Adipocyte Targeting Sequence (ATS)

An ATS peptide capable of targeting prohibitin is Cys Lys Gly Gly Arg Ala Lys Asp Cys (CKGGRAKDC). Using a solid fluoreonylmethoxycarbonyl (Fmoc) peptide synthesis method, the ‘CKGGRAKDC’ monomer peptide was synthesized. For this, a synthesis method that increases individual amino acids one by one according to a given amino acid sequence was used. After the elongation of the peptide chain was completed, the resulting product was treated with trifluoroacetic acid (TFA), thereby obtaining a free peptide.

### Preparation Example 2: Preparation of ATS-PEG-NH₂

The ATS peptide contains Cys at both ends, exposing a thiol group. A maleimide-PEG-NH₂ (M.W. 1,000˜5,000 Da) polymer was used to subsequently bind the ATS peptide to PLGA nanoparticles. The ATS peptide was reacted with acetic anhydride at room temperature for 1 hour to acetylate the N-terminus of the peptide and protect it from the reaction. Afterwards, a desalting process was performed in PBS to remove unreacted acetic anhydride. Afterwards, like maleimide-PEG-NH₂, the ATS peptide and the maleimide-PEG-NH₂ (M.W. 1,000˜5,000) were reacted in a molar ratio of 1:1 at 4° C. for 12 to 24 hours to form a thioester bond between maleimide and a thiol group. For purification and subsequent buffer exchange, the prepared ATS-PEG-NH₂ may be purified using size exclusion chromatography.

### Preparation Example 3: Preparation of PLGA NPs

Both 3 mg of a heme oxygenase-1 inducer (Hemin or CoPP) and 125 mg of PLGA (Resomer 502H) were dissolved in 2.5 mL of DMSO, dripped into 20 mL of a 4% (w/v) polyvinyl alcohol (PVA) aqueous solution and slowly stirred for 1 hour, thereby forming PLGA nanoparticles. To disperse small vesicles, the solution was homogenized by sonication at 20% amplitude for 20 seconds and stirred at room temperature for 6 hours in a fume hood until all of the organic solvent evaporated. The organic solvent was evaporated to produce small PLGA nanoparticles with a size of approximately 200 nm, and the nanoparticles were rapidly frozen using liquid nitrogen and then lyophilized under vacuum conditions at −40° C. to be dissolved in a 0.1 M MES buffer.

### Example 1: Preparation of ATS/PLGA NPs

The PLGA nanoparticles were dissolved in a 0.1 M MES buffer, reacted with 0.3 mM EDC for 30 minutes, and then reacted with 0.15 mM suflo-NHS for 2 hours to perform an EDC/NHS substitution reaction. Afterwards, ATS-PEG-NH₂ was added in a molar ratio of 0.5 to 2 with respect to PLGA NPs and reacted at room temperature for 3 hours to covalently bond ATS to PLGA NPs.

The liver/adipose tissue dual-targeting composite nano-drug carrier prepared in the above way was separated by centrifugation at 4° C. and 20,000 x G, rapidly frozen using liquid nitrogen, and then lyophilized under a vacuum condition at −40° C.

### Experimental Example 1: Comparison of Encapsulation Efficiency According to PLGA Molecular Weights and PVA Concentrations

3 mg of heme oxygenase-1 inducer and 125 mg of PLGA were dissolved in 2.5 mL of DMSO, dipped in each concentration of PVA aqueous solution, and stirred for 1 hour, thereby forming PLGA nanoparticles. The solution was homogenized by sonication for 1 minute, the organic solvent was evaporated to form PLGA nanoparticles, and then the PLGA nanoparticles were evaporated. 1 mg of the PLGA nanoparticles was dissolved in an organic solvent in which dichloromethane and acetonitrile were mixed at a ratio of 2:1, and then absorbance was measured at 580 to 630 nm to measure a drug amount.

TABLE 1

| PLGA (50:50) | | |
|---|---|---|
| molecular weight | PVA | Encapsulation |
| (Mw) | concentration | efficiency |
| 7,000 to 17,000 | 2% (w/v) | 3.5 ± 0.6% |
| | 4% (w/v) | 5.8 ± 0.8% |
| | 6% (w/v) | 3.8 ± 0.8% |
| 24,000 to 38,000 | 2% (w/v) | 2.5 ± 0.6% |
| | 4% (w/v) | 2.1 ± 0.5% |
| | 6% (w/v) | 1.4 ± 0.3% |

As shown in Table 1, as a result of measuring encapsulation efficiency in nanoparticles according to a PLGA molecular weight and a PVA concentration, it was confirmed that the maximum yield of encapsulation efficiency was obtained when the PLGA polymer with a molecular weight of 7,000 to 17,000 and 4% (w/v) PVA were used.

### Experimental Example 2: Confirmation of Particle Size of ATS/PLGA NPs

Nanoparticle sizes according to the molecular weight of maleimide-PEG-NH₂ and the molar ratio of ATS-PEG-NH₂ and the PLGA nanoparticles were measured.

3 mg of a heme oxygenase-1 inducer (Hemin or CoPP) was dissolved in both 125 mg of PLGA and 2.5 mL of DMSO, dripped into a different concentration of a 4% (w/v) polyvinyl alcohol (PVA) aqueous solution, and slowly stirred for 1 hour, thereby forming nanoparticles. The solution was homogenized by sonication and then the organic solvent was evaporated to form PLGA nanoparticles, and then the nanoparticles were lyophilized. The PLGA nanoparticles were dissolved in a 0.1 M MES buffer, reacted with 0.3 mM EDC for 30 minutes, and 30 minutes later, reacted with 0.15 mM suflo-NHS for 2 hours to prepare an EDC/NHS substitution reaction. Afterwards, the prepared ATS-PEG-NH₂ was added in a certain molar ratio and reacted with linkers with different molecular weights at room temperature for 3 hours to allow the ATS-PEG-NH₂ to covalently bind to the PLGA nanoparticles. The completed liver/adipose tissue dual-targeting nano drug delivery system was isolated by centrifugation, lyophilized, and stored. The size and zeta potential of the nanoparticles were diluted in distilled water and analyzed using a zeta-sizer (Malvern).

TABLE 2

| | | Particle size of | |
|---|---|---|---|
| Molecular weight | ATS-PEG-NH2:PLGA | composite nano-drug | |
| of linker | NPs (M) | carrier | |
| 1000 Da | 0.5:1 | 235.1 ± 2.5 | nm |
| | 1:1 | 244.7 ± 12.1 | nm |
| | 2:1 | 268.1 ± 24.5 | nm |
| 2000 Da | 0.5:1 | 282.1 ± 17.9 | nm |
| | 1:1 | 293.1 ± 21.5 | nm |
| | 2:1 | 311.1 ± 19.9 | nm |
| 3000 Da | 0.5:1 | 282.1 ± 28.3 | nm |
| | 1:1 | 302.6 ± 34.5 | nm |
| | 2:1 | 323.5 ± 35.2 | nm |
| 5000 Da | 0.5:1 | 322.8 ± 47.5 | nm |
| | 1:1 | 362.9 ± 51.5 | nm |
| | 2:1 | 382.1 ± 44.2 | nm |

As shown in Table 2, when ATS and the PLGA nanoparticles were combined using 1000 Da maleimide-PEG-NH₂, nanoparticles with a uniform distribution in the mid-200 nm range could be obtained. When using a linker with a higher molecular weight, the size and standard distribution of the nanoparticles increase, but the optimal particle size is 200 to 400 nm due to the maximized targeting ability and manufacturing uniformity of the nanoparticles (in subsequent targeting and efficacy experiments, comparison experiments were conducted only with 1000 Da and 2000 Da).

### Experimental Example 3: Confirmation of Drug-Specific Delivering Ability

The comparison in in vitro targeting ability to 3T3L1 (adipocytes), Raw264.7 (macrophages), and HepG2 (liver cells) was performed by the molecular weights (1000 Da and 2000 Da) of the linker (maleimide-PEG-NH₂) and the molar ratios of PLGA nanoparticles upon covalent bonding of ATS-PEG-NH₂ to which the maleimide-PEG-NH₂ and ATS peptide bound (ATS-PEG-NH2: PLGA=0.5:1, 1.5:1, 2:1).

After 3T3L1 (adipocytes), Raw264.7 (macrophages), and HepG2 (liver cells) were treated with a Cy5.5 fluorescence-loaded composite nano-drug carrier for 4 hours, the cells were cultured for 18 hours. Afterwards, the amount of fluorescence per cell was quantified using flow cytometry for each type of cells using mean fluorescence intensity.

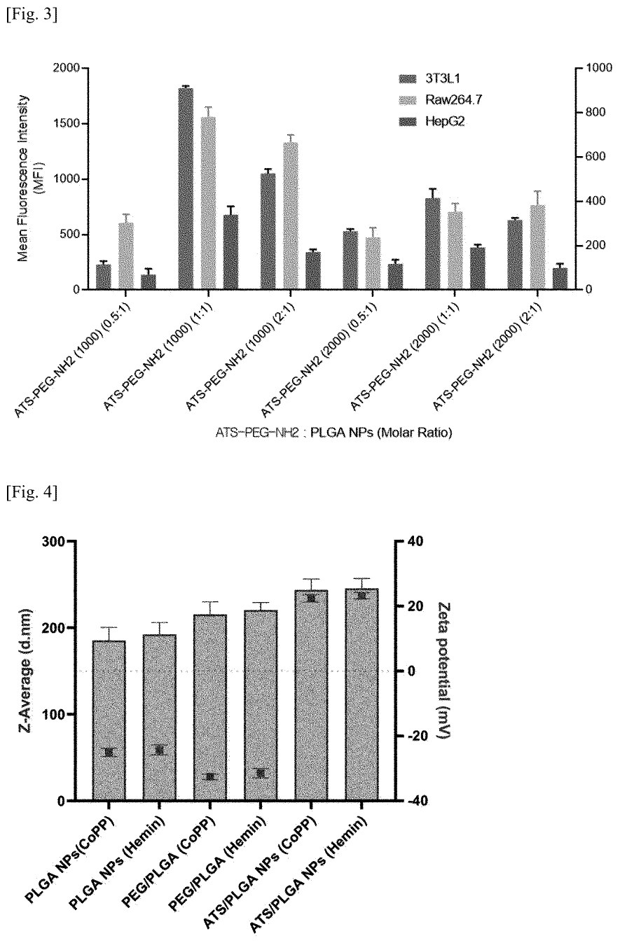

As a result, the most excellent drug-specific delivering ability was confirmed with the linker of 1000 Da in the ATS-PEG-NH₂:PLGA molar ratio of 1:1, and the corresponding composite nano-drug carrier was selected in all experiments according to Examples of the present invention (FIG. 3).

### Experimental Example 4: Characterization of Liver/Adipose Tissue Dual-Targeting Nano-Drug Carrier

The composite nano-drug carrier (1 mg/mL) manufactured in Example 1 was diluted in DMSO to analyze covalent bonding using VNMRS 600 MHZ (VARIAN). The particle size and zeta potential of the composite nano-drug carrier were analyzed using a zeta-sizer (Malvern) after being diluted in distilled water.

FIG. 4 shows the result that shows the zeta potential and nanoparticle size of the composite nano-drug carrier ATS/PLGA NPs manufactured in Example 1. The sizes of PEG/PLGA NPs (no ATS) and ATS/PLGA NPs were larger than PLGA NPs, and for ATS/PLGA NPs, the surface charge increased to a positive value due to the positive charge of the peptide.

### Experimental Example 5: Release Behavior Experiment of Liver/Adipose Tissue Dual-Targeting Nano Drug Delivery System

PBS containing a drug released from the nanoparticles was obtained by centrifuging the composite nano-drug carrier manufactured in Example 1 was centrifuged per time after being dissolved in PBS at 1 mg/mL, and absorbances were measured at 630 nm for hemin and 580 nm for CoPP in the corresponding samples to detect the hemin and CoPP amounts released in PBS. To simulate the environment in blood, serum was added to PBS at a volume ratio of 10%.

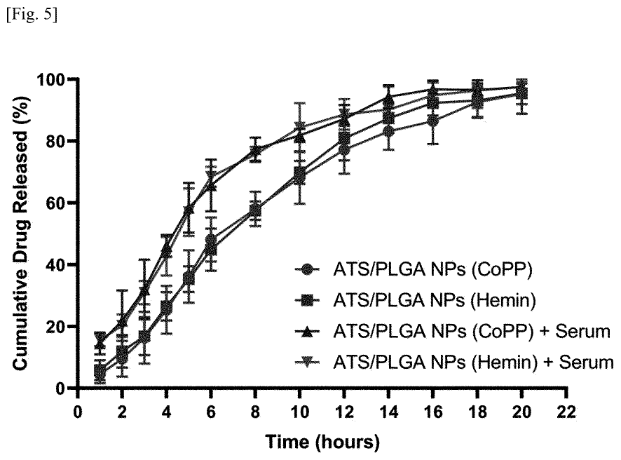

The results are shown in FIG. 5, and the uniform drug release behavior was confirmed for 24 hours, and the stability in blood was confirmed by confirming the uniform drug release behavior even in a state containing serum.

### Experimental Example 6: Construction of Mouse Models of Obesity-Induced Type 2 Diabetes

C57BL/6J mice were purchased from Orient Bio Inc. and acclimated for one week. From week 3, a 60% kcal high fat diet (HFD, Central Lab Animal, Inc.) was mixed with normal diet and fed. From week 6, only HFD was fed, and HFD was additionally fed for 8 weeks. In week 20, it was confirmed that the body weight reached 45 to 55 g, and the fasting blood glucose level also exceeded 250 mg/dl.

### Experimental Example 7: Construction of Mouse Models of Obesity-Induced Non-Alcoholic Steatohepatitis

C57BL/6J mice were purchased from Orient Bio Inc. and acclimated for one week. From week 3, a 60% kcal high fat diet (HFD, Central lab Animal, Inc.) was mixed with normal diet and fed, and mixed with 10% D-fructose in drinking water and fed. From week 6, only HFD was fed, and at the same time, a 25% D-fructose aqueous solution was given, and special feed and drinking water were maintained for 28 weeks. In week 30, it was confirmed that the body weight reached 55 to 60 g, and the fasting blood glucose level also exceeded 250 mg/dl.

### Experimental Example 8: In Vivo Distribution Experiment of Liver/Adipose Tissue Dual-Targeting Nano Drug Delivery System

After constructing the mouse models of obesity-induced type 2 diabetes and the mouse models of obesity-induced non-alcoholic steatohepatitis, Cy5.5 fluorescence-encapsulated ATS/PLGA NPs were intravenously injected once at a dose of 3 mg/kg. After 1, 4, and 24 hours, the mice were euthanized and the fluorescence of major organs was measured using VISQUE InVivo Smart.

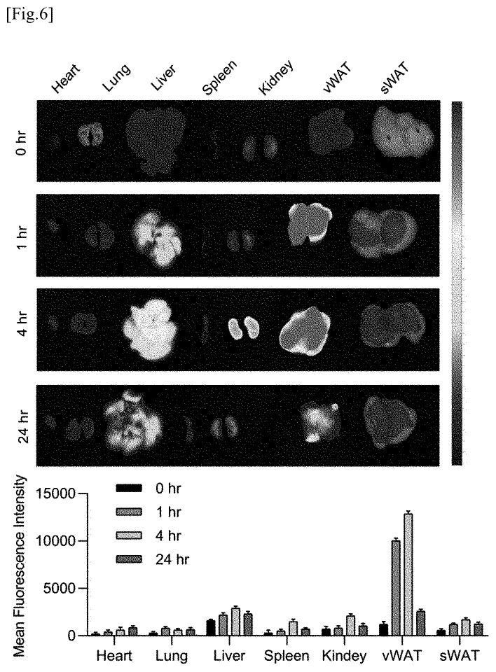

FIG. 6 shows the result of the time-dependent long-term distribution of ATS/PLGA NPs in animal models of obesity-induced type 2 diabetes, in the obesity-induced type 2 diabetes models, prohibitin expression was concentrated in vWAT (visceral fat), and accumulation in visceral fat was most prominent over time.

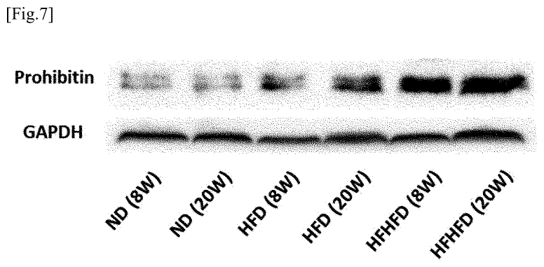

FIG. 7 shows the results of comparing protein expression levels by taking liver tissue from a normal diet (ND)-administered group, a 60% kcal high fat diet (HFD)-administered group for inducing animal models of obesity-induced type 2 diabetes, and a 60% kcal high fat diet+25% fructose (HFHFD)-administered group for inducing animal models of obesity-induced non-alcoholic steatohepatitis at week 8 and week 20. Liver tissue was homogenized in a RIPA buffer, isolated by molecular weight through electrophoresis, treated with anti-prohibitin antibodies or anti-GAPDH antibodies, and treated with secondary antibodies conjugated with a luminase to measure the expression levels. As a result, the expression level of prohibitin increased in liver tissue derived from fatty liver, confirming that the nanoparticles can target the liver with high efficiency.

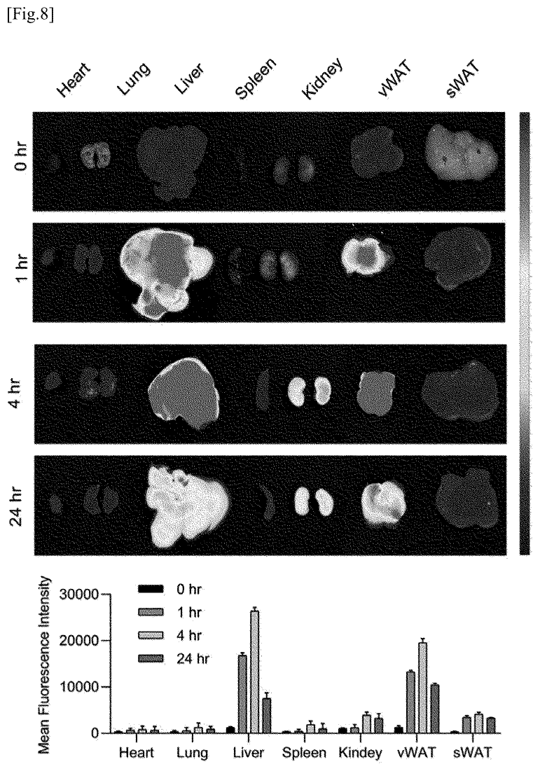

FIG. 8 shows the results of the time-dependent long-term distribution of ATS/PLGA NPs in animal models of obesity-induced non-alcoholic steatohepatitis, confirming that the nanoparticles are concentrated in the liver and visceral fat with high efficiency in the steatohepatitis models.

### Experimental Example 9: Effect of Liver/Adipose Tissue Dual-Targeting Nano Drug Delivery System in Treating Obesity And Obesity-Induced Metabolic Disease

After constructing the animal models of obesity-induced type 2 diabetes and the animal models of obesity-induced non-alcoholic steatohepatitis, ATS/PLGA NPs in which 1 mg/kg of heme oxygenase-1 inducer was encapsulated were injected intravenously once a week for 4 weeks. Here, the results of a control administered intravenously with ATS/PLGA NPs (vehicle) in which 1 mg/kg of heme oxygenase-1 inducer was not encapsulated once a week for 4 weeks and a control administered intraperitoneally with 0.25 mg/kg of heme oxygenase-1 inhibitor (ZnPP) once a week for 4 weeks while intravenous administration with ATS/PLGA NPs in which 1 mg/kg of heme oxygenase-1 inducer was encapsulated once a week for 4 weeks were compared. The body weight was measured for 7 weeks, and then the mice were dissected to obtain mRNA and proteins from major organs such as the liver and adipose tissue for biomarker analysis.

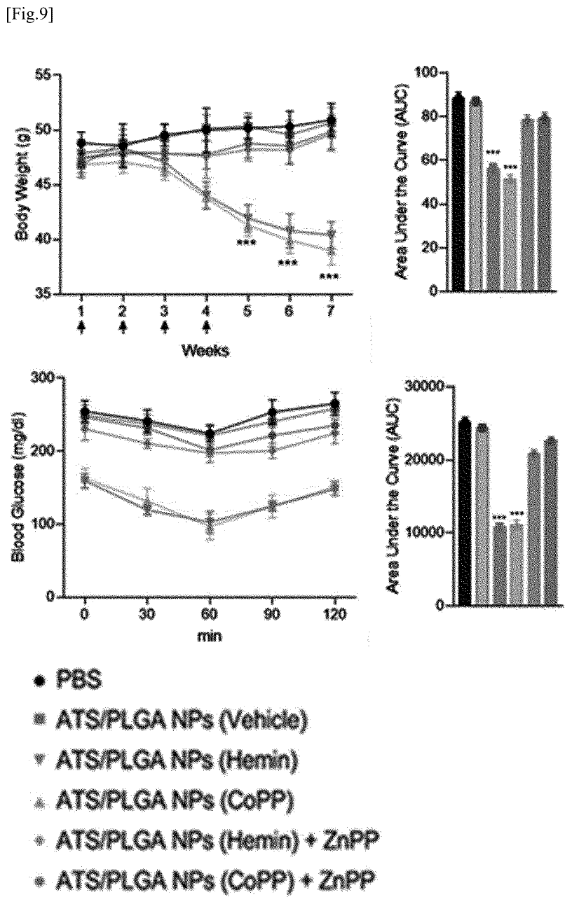

FIG. 9 shows the results of evaluating the change in body weight by week after 4-week administration of ATS/PLGA NPs and insulin resistance 3 weeks after the end of administration in animal models of obesity-induced type 2 diabetes. Body weights of the two groups administered with the heme oxygenase-1 inducer decreased by approximately 20%, and there is no effect when the heme oxygenase-1 inhibitor (ZnPP) is co-administered. When insulin is administered intraperitoneally and blood glucose levels are checked for 2 hours, the two groups administered with the heme oxygenase-1 inducer exhibit the highest reactivity.

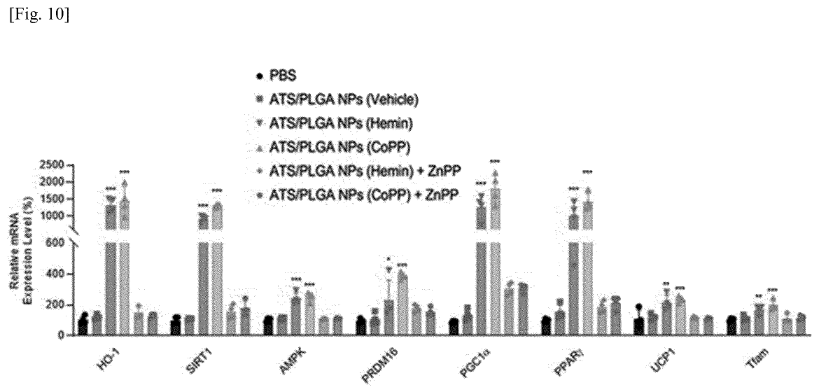

FIG. 10 shows the result of analyzing biomarkers in adipose tissue after 4-week administration of ATS/PLGA NPs in animal models of obesity-induced type 2 diabetes. In the two groups administered with the heme oxygenase-1 inducer, heme oxygenase-1 (HO-1) was elevated, and downstream genes (SIRT1 and AMPK), brown adipocyte differentiation markers (PRDM16, PPARγ, and PGC1α), and mitochondrial activity markers (UCP1 and Tfam) significantly increased.

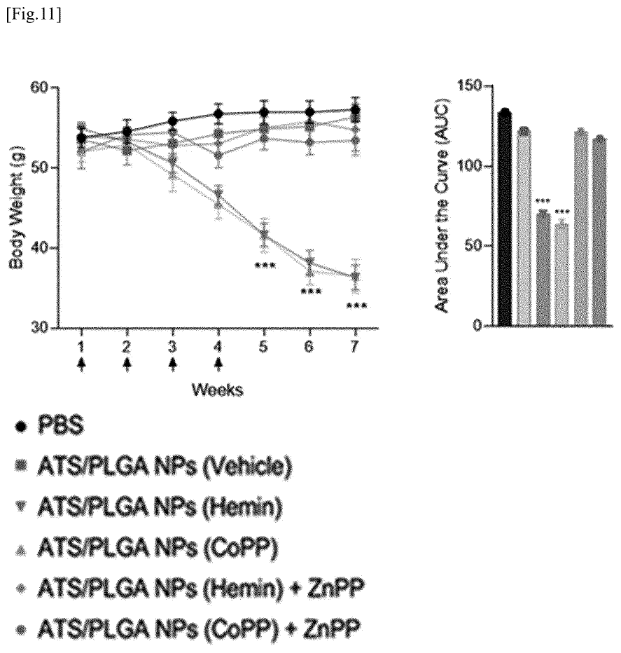

FIG. 11 shows the result of measuring the change in body weight by week after 4 weeks of intravenous administration of ATS/PLGA NPs in animal models of obesity-induced non-alcoholic steatohepatitis.

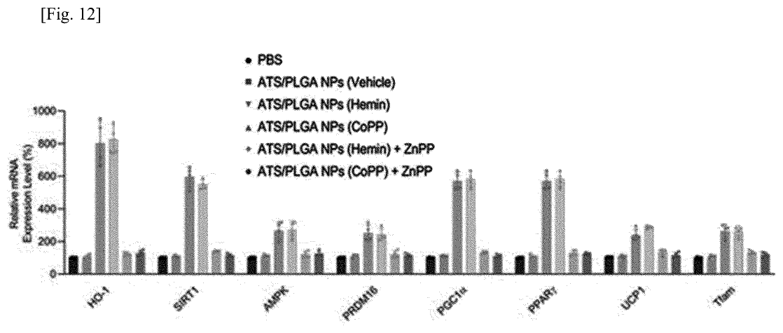

FIG. 12 shows the result of analyzing biomarkers in adipose tissue after 4-week intravenous administration of ATS/PLGA NPs in animal models of obesity-induced non-alcoholic steatohepatitis. In the two groups administered intravenously with the heme oxygenase-1 inducer, heme oxygenase-1 (HO-1) was elevated, and the downstream genes (SIRT1 and AMPK), the brown adipocyte differentiation markers (PRDM16, PPARγ, and PGC1α), and the mitochondrial activity markers (UCP1 and Tfam) significantly increased.

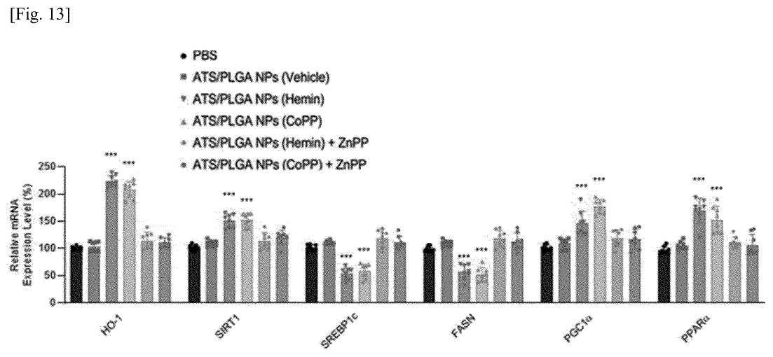

FIG. 13 shows the result of analyzing biomarkers in liver tissue after 4-week intravenous administration of ATS/PLGA NPs in animal models of obesity-induced non-alcoholic steatohepatitis. In the two groups administered intravenously with the heme oxygenase-1 inducer, it was confirmed that heme oxygenase-1 (HO-1) was elevated, the downstream gene (SIRT1) and mitochondrial activity markers (PPAR α and PGC1α) were significantly elevated, and lipid storage-associated biomarkers (SREBP1c and FASN) decreased.

This means that ATS/PLGA NPs in which the heme oxygenase-1 inducer was encapsulated induce differentiation of brown adipocytes in adipose tissue, activating mitochondrial function and energy conversion of fatty acids in the animal models of obesity-induced type 2 diabetes and the animal models of obesity-induced non-alcoholic steatohepatitis. This shows that it can effectively suppress fatty acids accumulated in the body and overcome obesity and obesity-induced metabolic diseases through weight loss. In addition, in the animal models of non-alcoholic steatohepatitis, the heme oxygenase-1 inducer has been shown to alleviate fatty liver symptoms by activating mitochondrial function in liver tissue and inhibiting fatty acid storage.

### Experimental Example 10: Analysis of Immune Cell Differentiation in Organs

After 4-week intravenous administration to the animal models of obesity-induced type 2 diabetes and the animal models of obesity-induced non-alcoholic steatohepatitis, immune cells were isolated from adipose tissue and liver tissue, and the differentiation of the immune cells was measured using flow cytometry.

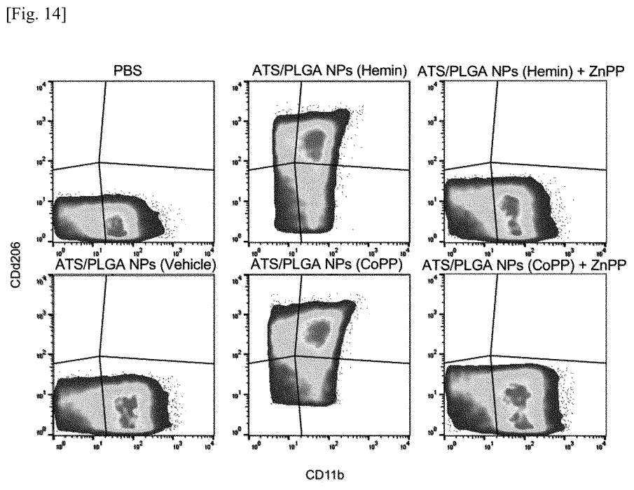

FIG. 14 shows the results of analyzing M2 phenotype differentiation of adipose tissue macrophages after 4-week administration of ATS/PLGA NPs in animal models of obesity-induced type 2 diabetes, confirming that the biomarker of anti-inflammatory macrophages, CD206+population, increased in the two groups administered with the heme oxygenase-1 inducer.

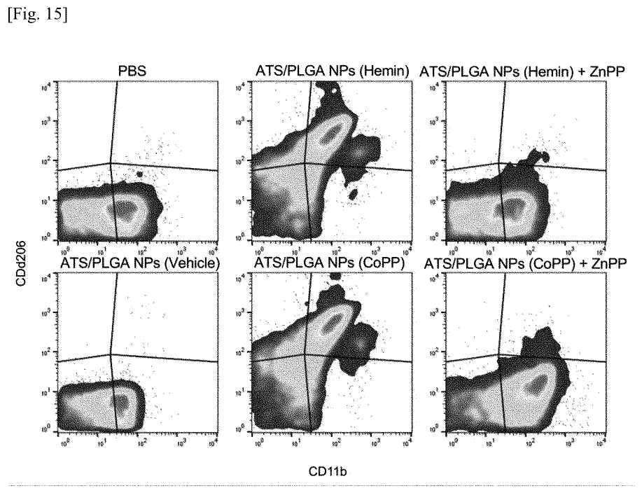

FIG. 15 shows the results of analyzing M2 phenotype differentiation of adipose tissue macrophages after 4-week administration of ATS/PLGA NPs in animal models of obesity-induced non-alcoholic steatohepatitis, confirming that the biomarker of anti-inflammatory macrophages, CD206+population, increased in the two groups administered with the heme oxygenase-1 inducer.

### Experimental Example 11: Analysis of Lipid Components in Organs and Blood

After 4-week intravenous administration to the animal models of obesity-induced type 2 diabetes and the animal models of obesity-induced non-alcoholic steatohepatitis, adipose tissue, liver tissue, and blood samples were obtained to separate a lipid layer through centrifugation. Afterwards, quantitative analysis for the lipid layer was performed using Free Fatty Acid Assay Kit-Quantification (ab65341), Triglyceride Assay Kit-Quantification (ab65336), and Cholesterol Assay Kit-HDL & LDL/VLDL (ab65390), produced by Abcam.

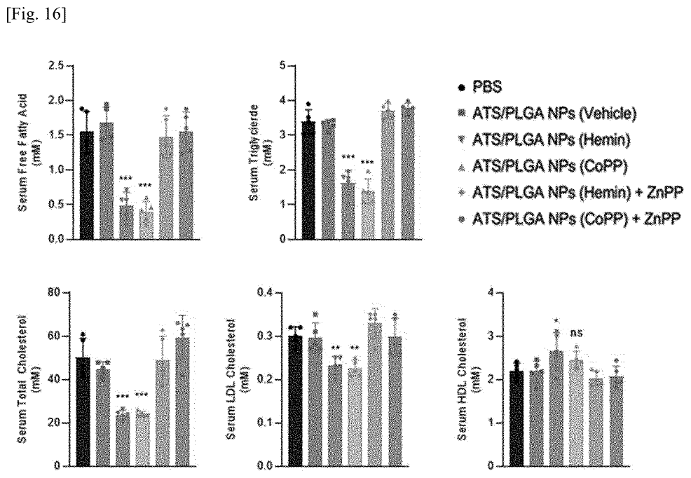

FIG. 16 shows the results of analyzing blood lipid composition after 4-week intravenous administration of ATS/PLGA NPs in animal models of obesity-induced type 2 diabetes. In the two groups administered with the heme oxygenase-1 inducer, blood fatty acid, triglyceride, and cholesterol levels were reduced.

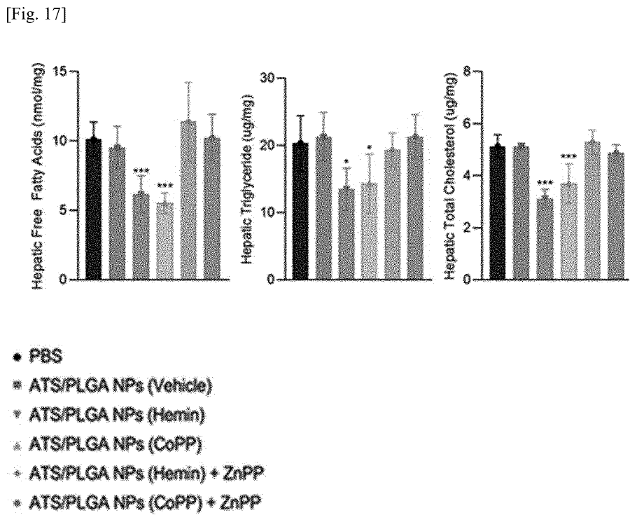

FIG. 17 shows the results of analyzing lipid composition in liver tissue after 4-week intravenous administration of ATS/PLGA NPs in animal models of obesity-induced non-alcoholic steatohepatitis. In the two groups administered with the heme oxygenase-1 inducer, blood fatty acid, triglyceride, and cholesterol levels were reduced.

### Experimental Example 12: Analysis of Inflammatory Cytokines in Organs and Blood

After 4-week intravenous administration to the models of obesity-induced type 2 diabetes, the models of obesity-induced type 2 diabetes, and the models of obesity-induced non-alcoholic steatohepatitis, adipose tissue and liver tissue, blood samples were obtained to isolate proteins. Afterwards, quantitative analysis for the proteins was performed using an ELISA kit (Invitrogen).

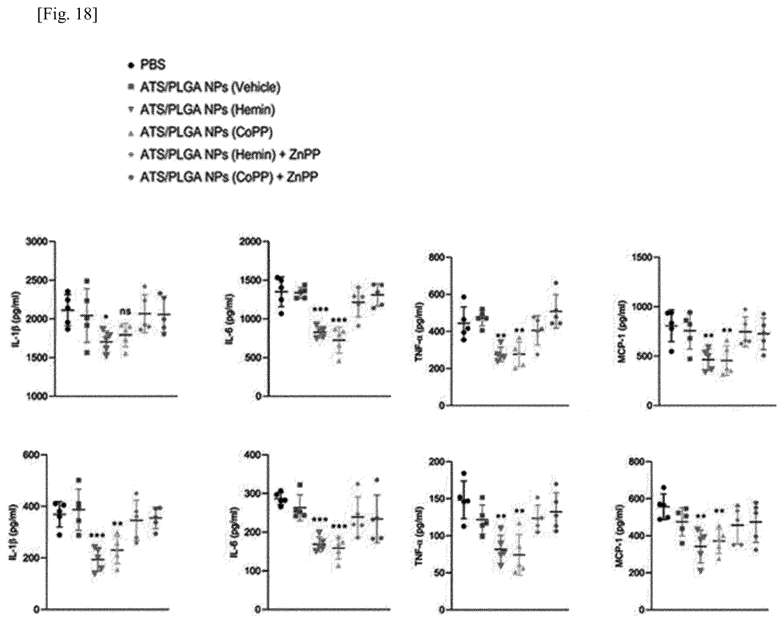

FIG. 18 shows the results of analyzing the amount of inflammatory cytokine proteins in adipose tissue (top) and blood (bottom) after 4-week intravenous administration of ATS/PLGA NPs in animal models of obesity-induced type 2 diabetes.

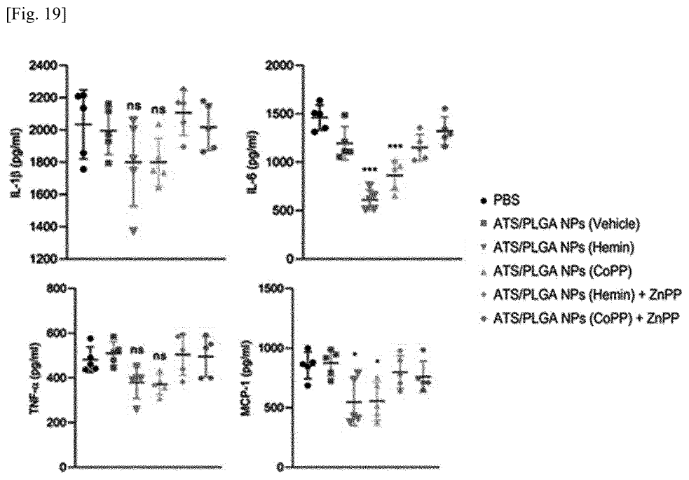

FIG. 19 shows the results of analyzing the expression levels of inflammatory cytokines in liver tissue after 4-week intravenous administration of ATS/PLGA NPs in animal models of obesity-induced non-alcoholic steatohepatitis.

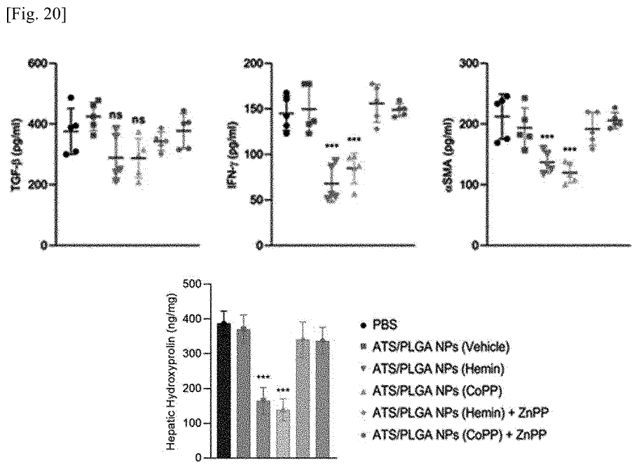

FIG. 20 shows the results of analyzing the expression levels of biomarkers (TGF-beta, IFNr, aSMA, and hydroxyproline) associated with the development of cirrhosis in liver tissue after 4-week intravenous administration of ATS/PLGA NPs in animal models of obesity-induced non-alcoholic steatohepatitis.

The heme oxygenase-1 inducer delivered to the adipose tissue promoted differentiation of white adipocytes and brown adipocytes to activate mitochondrial function. This causes fatty acid depletion, lowering fatty acid concentration in adipose tissue and blood. The reduction in fatty acids showed a weight loss effect and a decrease in fatty acid reduction in other organs.

Moreover, the liver/adipose tissue dual-targeting nano drug delivery system can simultaneously act on adipose tissue and fatty liver, thereby alleviating symptoms of fatty liver caused by obesity and obesity-induced metabolic diseases.

The heme oxygenase-1 inducer delivered to liver tissue showed an anti-inflammatory effect and a fat accumulation inhibitory effect in liver tissue.

In addition, the degrees of inflammation and cirrhosis occurring in fatty liver were alleviated by inducing differentiation of anti-inflammatory M2 phenotype of macrophages in liver tissue.

1. A liver/adipose tissue dual-targeting composite nano-drug carrier, comprising:
drug-containing poly(L-lactide-co-glycolide) (PLGA) nanoparticles; and
an adipocyte targeting sequence (ATS) peptide.

2. The composite nano-drug carrier of claim 1, wherein the drug is a heme oxygenase-1 inducer.

3. The composite nano-drug carrier of claim 1, wherein the ATS peptide is capable of targeting prohibitin.

4. The composite nano-drug carrier of claim 1, wherein the ATS peptide is represented by SEQ ID NO: 1.

5. The composite nano-drug carrier of claim 1, wherein the ATS peptide binds to the surface of the drug-containing PLGA nanoparticles using a linker.

6. The composite nano-drug carrier of claim 2, wherein the heme oxygenase-1 inducer is cobaltic protoporphyrin IX chloride (CoPP) or hemin.

7. The composite nano-drug carrier of claim 5, wherein the linker is maleimide-PEG-amine.

8. The composite nano-drug carrier of claim 1, wherein the average molecular weight of PLGA ranges from 5,000 to 18,000.

9. The composite nano-drug carrier of claim 7, wherein the average molecular weight of the linker ranges from 1,000 to 5,000.

10. The composite nano-drug carrier of claim 7, wherein the average particle size ranges from 200 to 400 nm.

11. The composite nano-drug carrier of claim 1, wherein the PLGA nanoparticles and the ATS peptide are bound together in a molar ratio of 1:0.5 to 2.

12. A method of preparing a liver/adipose tissue dual-targeting composite nano-drug carrier, comprising:
combining an adipocyte targeting sequence (ATS) peptide with drug-containing poly(L-lactide-co-glycolide) (PLGA) nanoparticles using linkers.

13. The method of claim 12, comprising:
preparing drug-containing PLGA nanoparticles by dissolving the drug and PLGA, stirring the resulting mixture, and adding a PVA aqueous solution thereto;
preparing the ATS peptide modified with maleimide-PEG-amine by acetylating the N-terminal of the ATS peptide and reacting it with the maleimide-PEG-amine; and
reacting the drug-containing PLGA nanoparticles through an NHS/EDC substitution reaction, and reacting the resulting substitution with the maleimide-PEG-amine-modified ATS peptide at room temperature for 2 to 4 hours to combine the ATS peptide with the drug-containing PLGA nanoparticles using linkers.

14. (canceled)

15. The method of claim 13, wherein the concentration of the PVA aqueous solution is 3 to 5% (w/v).

16.-18. (canceled)

19. The method of claim 13, wherein the PLGA nanoparticles and the ATS peptide are bound in a molar ratio of 1:0.5 to 2.

20. A composite nano-drug carrier prepared by the method of claim 12.

21. A pharmaceutical composition for preventing or treating obesity or an obesity-induced metabolic disease, comprising the composite nano-drug carrier of claim 1 as an active ingredient.

22.-23. (canceled)

24. A food composition for preventing or improving obesity or an obesity-induced metabolic disease, comprising:
the composite nano-drug carrier of claim 1 as an active ingredient.

25. (canceled)

26. A method of preventing or treating obesity or an obesity-induced metabolic disease, comprising:
administering a therapeutically effective amount of the liver/adipose tissue dual-targeting composite nano-drug carrier of claim 1 to a subject.

27.-28. (canceled)


SEQUENCE LISTING (1 sequence)

SEQ ID NO: 1 - protein, 9 aa, synthetic construct
CKGGRAKDC